(19) United States
(12) Patent Application Publication
(10) Pub. No.: US 2026/0207059 A1
(43) Pub. Date: Jul. 23, 2026

(54) SYSTEMS AND METHODS TO PROVIDE HAPTIC FEEDBACK ON CLOT ENGAGEMENT

(71) Applicant: TERUMO CORPORATION, Tokyo (JP)

(72) Inventors: Dasnurkar Anup, Rancho Santa Margarita, CA (US); Melissa Buzby, Long Beach, CA (US)

(21) Appl. No.: 19/124,029

(22) PCT Filed: Oct. 25, 2023
(86) PCT No.: PCT/US2023/035917

§ 371 Date: Apr. 24, 2025

Related U.S. Application Data
(60) Provisional application No. 63/419,475, filed on Oct. 26, 2022.

(51) Int. Cl. A61B 5/02; A61B 5/00; A61B 5/05; A61B 90/00

(52) U.S. Cl. CPC A61B 5/02007; A61B 5/0084; A61B 5/05; A61B 5/6852; A61B 5/7405; A61B 90/06; A61B 2560/0462

(57) ABSTRACT

Disclosed are example embodiments of a catheter system for blood clot detection. The catheter system for blood clot detection includes a catheter having a proximal end and a distal end. The catheter system for blood clot detection also includes a blood clot sensor coupled to the distal end of the catheter. Additionally, the catheter system for blood clot detection includes a processor configured to process signals from the blood clot sensor. The blood clot sensor may include magnetoelastic sensor. The blood clot sensor may include a light-based sensor. The blood clot sensor may include a ultrasound sensor.

### CROSS-REFERENCE TO RELATED APPLICATIONS

The present is a national phase entry of International Application No. PCT/US23/35917, filed Oct. 25, 2023, which claims priority to U.S. Provisional Application No. 63/419,475 entitled “SYSTEMS AND METHODS TO PROVIDE HAPTIC FEEDBACK ON CLOT ENGAGEMENT” filed on Oct. 26, 2022, both of which are hereby incorporated by reference in their entireties for all purposes.

### FIELD OF THE DISCLOSURE

The disclosure relates generally to the field of medical sensors, and specifically and not by way of limitation, some embodiments are related to medical sensors for the detection of blood clots.

### BACKGROUND

Intravascular procedures may typically use catheters within a patient's vasculature. Some procedures may use a catheter to remove blood clots. However, blood clots are not radio-opaque. In other words, blood clots are opaque to forms of radiation, such as X-rays or other forms of radiation. Radiopaque objects block radiation rather than allow the radiation to pass through. Accordingly, because blood clots are not radio-opaque, physicians may have to rely on forms of medical imaging other than medical X-rays. Some other forms of medical imaging may include but are not limited to computed tomography (CT) scan, angiograms (e.g., for visualizing the flow of injected contrast solution), or some other forms of medical imagine that do not use X-rays to properly identify the location of a blood clot within a vasculature. After the blood clot is located, the medical imaging may be used by the medical professional to engage the blood clot with a device of the medical professional's choice to remove the blood clot. The process of medical imaging and engaging a blood clot by a medical professional may have a variable success rate depending on a number of factors, including but not limited to, the disease state, anatomical limitations, imaging limitations, device limitations, and/or physician skill or experience.

For example, in devices that are related to treatment of pulmonary embolism, a patient may usually be awake and breathing. Accordingly, an accurate angiogram may be challenging due to patient movement, e.g., chest movement, lung movement, or other patient movements. Additionally, the catheters used for the removal of blood clots are generally large bore and may potentially dislodge the blood clot, e.g., when large pressures are used to inject contrast material into a patient's vasculature. The large pressures used to inject contrast material into a patient's vasculature may further create broken blood clots in the vasculature. The blood clots in the vasculature may move distally, or inadvertently push the blood clot towards an open artery increasing the risk to the patient. Additionally, without having a precise clot location, the extraction device may be far away from the blood clot, leading to a substantial amount of blood loss in an attempt to pull or aspirate the clot out of the vasculature. Accordingly, a need exists for a sensor-based mechanism that provides a user with feedback to know that a blood clot is either engaged with a device or the blood clot is near enough so removal of the blood clot may be effective.

### SUMMARY

In one example implementation, an embodiment includes a sensor-based mechanism that provides a user with feedback to know that a blood clot is either engaged with a device or the blood clot is near enough so removal of the blood clot may be effective.

Disclosed are example embodiments of a catheter system for blood clot detection. The catheter system for blood clot detection includes a catheter having a proximal end and a distal end. The catheter system for blood clot detection also includes a blood clot sensor coupled to the distal end of the catheter. Additionally, the catheter system for blood clot detection includes a processor configured to process signals from the blood clot sensor.

The features and advantages described in the specification are not all-inclusive. In particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the disclosed subject matter.

For the purposes of the terminology described herein, the terms clot, thrombus, embolus, and obstruction can be used synonymously.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies. The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure. These and other aspects, features and advantages of which embodiments of the disclosure are capable of will be apparent and elucidated from the following description of embodiments of the present disclosure, reference being made to the accompanying drawings, in which:

FIG. 1 is a diagram illustrating an example catheter system for blood clot detection in accordance with the systems and methods described herein.

FIG. 2 is a diagram illustrating the example catheter system for blood clot detection of FIG. 1 with the tip of the catheter entering a patient's vasculature in accordance with the systems and methods described herein.

FIG. 3 is a diagram illustrating the example catheter system for blood clot detection including a magnetoelastic sensor that is emitting magnetic flux for the detection of blood clots in accordance with the systems and methods described herein.

FIG. 4 is a diagram illustrating the example catheter system for blood clot detection that uses light-based sensor technology for the detection of blood clots in accordance with the systems and methods described herein.

FIG. 5 is a diagram illustrating the example catheter system for blood clot detection that uses ultrasound sensors for the detection of blood clots in accordance with the systems and methods described herein.

FIG. 6 is a diagram illustrating an example catheter system for blood clot detection including a durometer gauge in accordance with the systems and methods described herein.

FIG. 7 is a diagram illustrating an example catheter system for blood clot detection including a density meter in accordance with the systems and methods described herein.

FIG. 8 is a diagram illustrating an example catheter system for blood clot detection including a flow meter in accordance with the systems and methods described herein.

FIG. 9 is a flow diagram illustrating the example method in accordance with the systems and methods described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

As discussed above, a need exists for a sensor-based mechanism that provides a user with feedback to know that a blood clot is either engaged with a device or the blood clot is near enough so removal of the blood clot may be effective. Some embodiments described herein may include one or more blood clot sensors mounted on a catheter tip. The blood clot sensor (or sensors) may provide feedback to a user when the tip of the catheter is near a blood clot or inside a blood clot. In one embodiment, the blood clot sensor (or sensors) may be a magnetoelastic sensor (or sensors). In another embodiment, the blood clot sensor (or sensors) may be a light-based sensor (or sensors). In yet another embodiment, the blood clot sensor (or sensors) may be an ultrasound sensor (or sensors).

An example embodiment may have a sensor/mechanism attached to a distal end of an aspiration catheter. The sensors or mechanisms may have the ability to provide feedback only when a clot is engaged, and not indicate to the presence of blood or a vascular wall.

The use of specialized sensors in catheters may enhance the accuracy of thrombectomy procedures. By offering direct feedback on clot engagement, these sensors may help reduce unintended interactions with the vascular wall or flowing blood. This specificity may not only streamlines the procedure but may also minimize potential tissue damage. As a result, the sensors may, in some embodiments, lead to more efficient clot removal and improved patient outcomes.

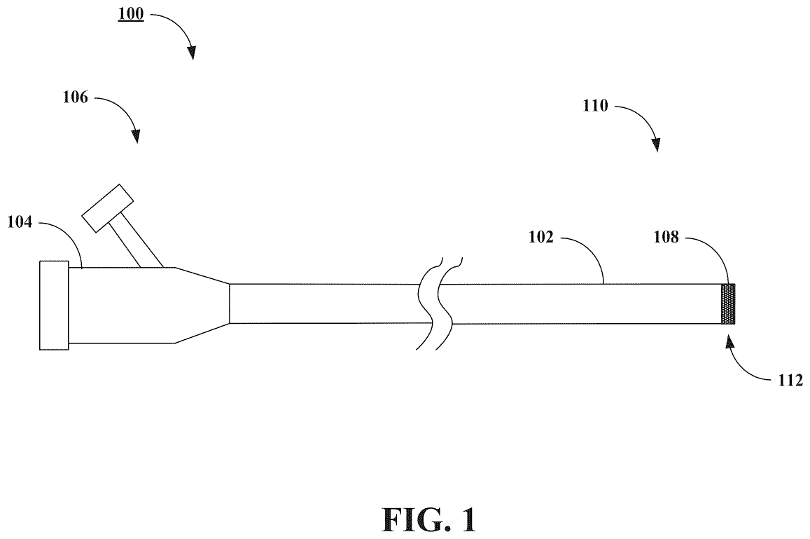

FIG. 1 is a diagram illustrating an example catheter system 100 for blood clot detection in accordance with the systems and methods described herein. The example catheter system 100 includes a catheter 102, a y-connector hub 104 at a proximal end 106 of the example catheter system 100, and a blood clot sensor (or sensors) 108 at the distal end 110 of the example catheter system 100. For example, the blood clot sensor 108 may be mounted on a tip 112 of the catheter 102.

In some embodiments described herein, the blood clot sensor(s) 108 may be a magnetoelastic sensor mounted on the tip 112 of the catheter 102. Similarly, in some embodiments described herein, the blood clot sensor 108 may use light-based sensor technology on the tip 112 of the catheter 102 to sense blood clots. In other embodiments described herein, the blood clot sensor 108 may use ultrasound sensors on the tip 112 of the catheter 102 to sense blood clots. In other embodiments, multiple sensors 108 may be located on a single tip 112 of the catheter 102, and the multiple sensors 108 may be of the same type or different types (e.g., light-based sensor with ultrasound sensor). In some embodiments, some tips 112 may have multiple sensors. Some example systems may include multiple catheters.

Referencing back to FIG. 1, the Y-connector hub 104 may serve as an interface, facilitating both the introduction of medical instruments into the catheter 102 and the withdrawal of fluids. The design of the Y-connector hub 104 may help ensure secure attachment and may minimize the risk of any disconnections during the procedure. The catheter 102, while depicted in a simplified form in FIG. 1, may come in various lengths and diameters suitable for different medical applications. The choice of material for catheter 102 may provide flexibility while ensuring structural integrity, thus allowing smooth navigation through the vascular system. Additionally, while the blood clot sensor(s) 108 at the distal end 110 may be used for clot detection, the catheter 102 may also incorporate other features or sensors, not illustrated in FIG. 1, designed to enhance he catheter 102's functionality and the safety of the procedure.

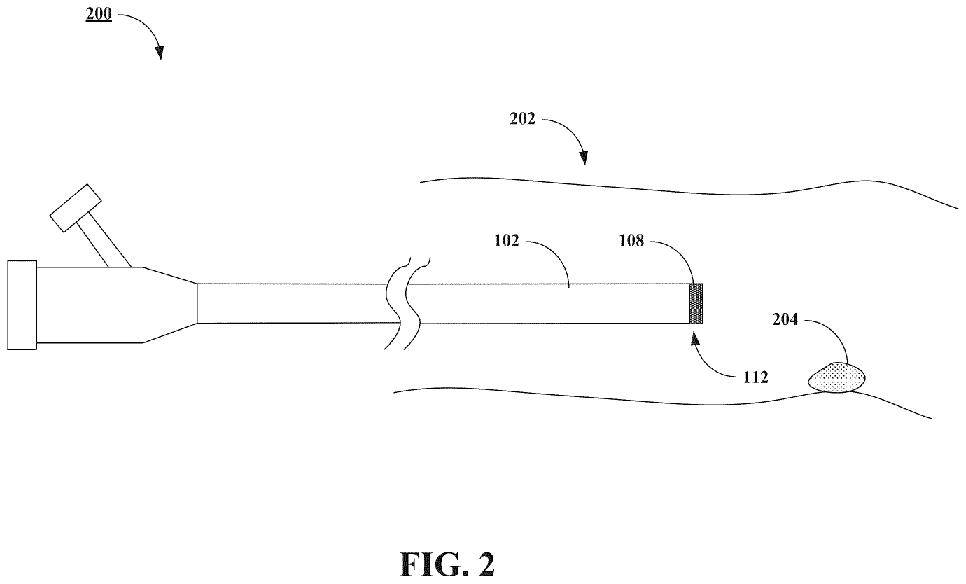

FIG. 2 is a diagram 200 illustrating the example catheter system of FIG. 1 with the tip 112 of the catheter 102 entering a patient's vasculature 202 in accordance with the systems and methods described herein. In the diagram 200, the blood clot sensor 108 (or sensors) (e.g., magnetoelastic sensor, light-based sensor, ultrasound sensor, or other sensor capable of sensing blood and/or a blood clot) may provide active feedback to a user when the tip 112 of the catheter 102 is near a blood clot 204 or inside a blood clot 204.

In FIG. 2, as the catheter system's tip 112 navigates through the patient's vasculature 202, in an example embodiment, the precise positioning and sensitive nature of the blood clot sensor(s) 108 may enable real-time monitoring of the catheter's proximity to potential obstructions, such as blood clot 204. This proactive monitoring may serve as a crucial safety feature, that may ensure that medical practitioners have continuous awareness of the catheter's environment, thereby aiding in effective clot engagement. Additionally, the responsive nature of the blood clot sensor(s) 108 may help distinguish between the presence of a blood clot 204 and surrounding vascular tissue. This distinction may help avoid unnecessary interventions and ensure targeted treatment, further optimizing the procedure's success and patient safety. The feedback provided by the sensor(s) may be visual, auditory, or tactile, offering the practitioner various ways to interpret and react to the catheter's position relative to clot 204.

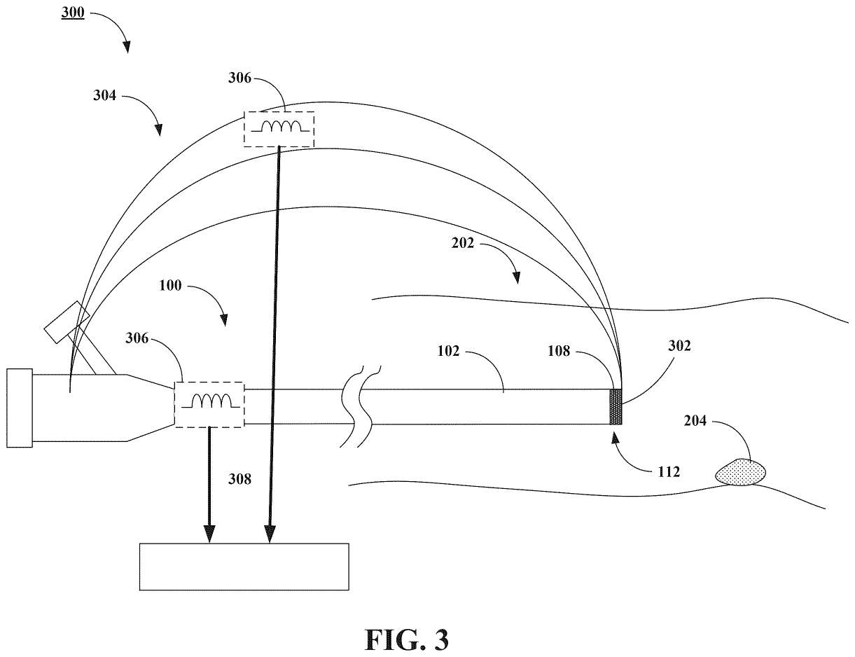

FIG. 3 is a diagram illustrating the example catheter system for blood clot detection 300 including a magnetoelastic sensor (or sensors) 302 that is emitting magnetic flux for the detection of blood clots in accordance with the systems and methods described herein. In the example embodiment, a magnetoelastic sensor 302 may emit magnetic flux 304. The magnetic flux 304 may be detected by a remotely located pick-up coil 306, for example, located outside of the patient's body so that no direct physical connections are required. In an example embodiment, the remotely located pick-up coil 306 may be external to the example catheter system 300, for example, located in an external console or the y-connector hub 104 or handle of the catheter 102. In another example embodiment, the remotely located pick-up coil 306 may be internal to the example catheter system 300. In an example embodiment a goal may be to have remote pickup coil (or other sensor) outside the patient body. Accordingly, the sensors may use a particular frequency. The particular frequency may respond based on what solid matter or fluid the sensor is close to, e.g., based on the location of intended use. Thus, a liquid would provide different response to the same frequency as opposed to a solid or semi-solid object. Some embodiments may be adjustable for different areas of use.

During blood coagulation, the viscosity of blood changes due to the formation of a soft fibrin clot. In turn, the change in blood viscosity shifts the characteristic resonance frequency of the magnetoelastic sensor 302 enabling real-time continuous monitoring of this biological event. By monitoring the signal output 308 as a function of time, a distinct blood clotting profile may be seen. The relatively low cost of the magnetoelastic ribbons may enable the use of magnetoelastic ribbons as disposable sensors. In some embodiments, the ability of the sensors to be disposable sensors may, along with a reduced volume of blood required, may make the magnetoelastic sensors 302 well suited for at-home and point-of-care testing devices.

Some embodiments may be used for applications in the treatment of pulmonary embolism. Additionally, fiber optic technology may also be used for the same purpose (e.g., pulmonary embolism). Pulmonary embolism is a blockage in one or more of the pulmonary arteries in a person's lungs. Pulmonary embolism may be caused by blood clots that may travel to the lungs from deep veins in the legs or, rarely, from veins in other parts of the body (deep vein thrombosis).

Some example embodiments of the systems and methods described herein may be used to remove blood clots that have traveled to the lungs, e.g., pulmonary embolisms. Some embodiments of the systems and methods described herein may be used to remove clots located in other parts of the body before those clots have a chance to travel to the lungs (or to other parts of the body). For example, some embodiments of the systems and methods described herein may be used to remove blood clots from veins in the legs before those blood clots have the chance to travel to the lungs.

Advanced iterations of the catheter system described herein may look to integrate a combination of sensor technologies to improve clot detection accuracy. In some embodiments, by leveraging the synergies of magnetoelastic, light-based, and ultrasound sensors, the system may triangulate and confirm clot presence with greater precision, reducing false positives. Furthermore, integrating real-time data analytics may enable healthcare providers to analyze the clot's characteristics in detail, such as its density and size, allowing for more targeted and effective interventions. This integration of technology may also pave the way for personalized treatment plans, wherein the catheter system adjusts its approach based on the unique clot profile of each patient. As medical technology continues to evolve, the potential for remote monitoring and telemedicine integration with the systems and methods described herein may also presents exciting prospects.

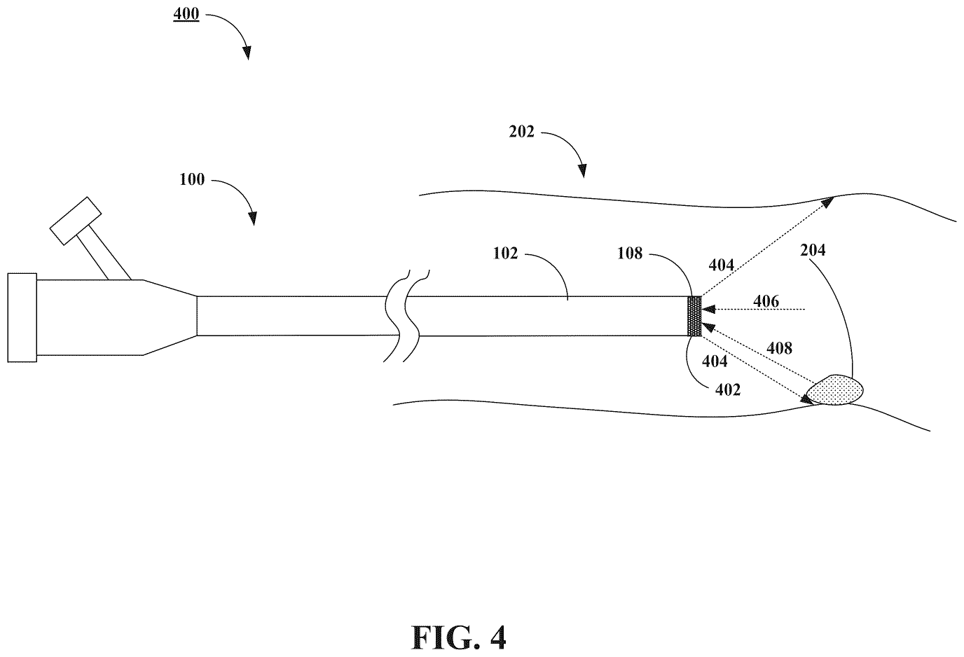

FIG. 4 is a diagram illustrating the example catheter system for blood clot detection 400 that uses light-based sensor technology for the detection of blood clots in accordance with the systems and methods described herein. The example embodiment of FIG. 4 may use light-based sensor technology. The light-based sensor technology may detect the difference in the wavelength, e.g., in nm, between blood and that of a solid object such as coagulated blood. For example, a light-based sensor (or sensors) 402 may include a sensor portion and a light emitting portion. The light emitting portion of the light-based sensor 402 may emit light 404. The light 404 emitted may be a broad beam in some embodiments. In other embodiments the light emitted 404 may be a narrower beam. Light 406 may be reflected back from blood in the vasculature. Light 408 may also be reflected back from a solid object such as coagulated blood in the vasculature, e.g., a blood clot 204 located in the vasculature. The light-based sensor 402 may sense the reflected light 406, which may have a different frequency depending on what is reflecting the reflected light 406. For example, blood clots may typically reflect light in a narrow range of wavelengths, and that range may be different from blood which typically reflects light at about 500 nm to about 700 nm. Accordingly, these differences in frequency may be used to tell the difference between blood clots and blood. While the example of FIG. 4 illustrates a light-based sensor 402 that includes a light source and a sensor, it will be understood that in other embodiments the light source and the sensor may be two distinct devices. Furthermore, it will be understood that some embodiments may include multiple sensors, multiple light sources or multiple combinations of sensors and light sources. Additionally, some embodiments may include combinations of individual sensors, individual light sources, and combined sensors and light sources.

The light source may be any transducer capable of emitting light, such as, for example, light emitting diodes (LEDs), lamps, or other light emitting devices. The sensor for Measuring the light reflected back may be any sensor capable of measuring light and providing a frequency or frequency range of the light received.

In certain embodiments, the precision and accuracy of the light-based sensor 402 may be enhanced by employing specific filters that allow only specific wavelengths of reflected light to pass through for detection. This may aid in clearly distinguishing between the wavelengths reflected by blood clots and those reflected by flowing blood. Furthermore, advanced algorithms may be integrated into the catheter system's software to interpret these reflected wavelengths more accurately, eliminating potential interferences or anomalies in the readings. For optimal performance, calibration techniques may be employed periodically to ensure the light-based sensors maintain their accuracy over time. With advancements in optoelectronics, future designs may also leverage more compact and efficient light sources, which may provide a broader spectrum of emitted light, which may improve the resolution of detected signals and enabling differentiation of even more subtle differences in the vascular environment.

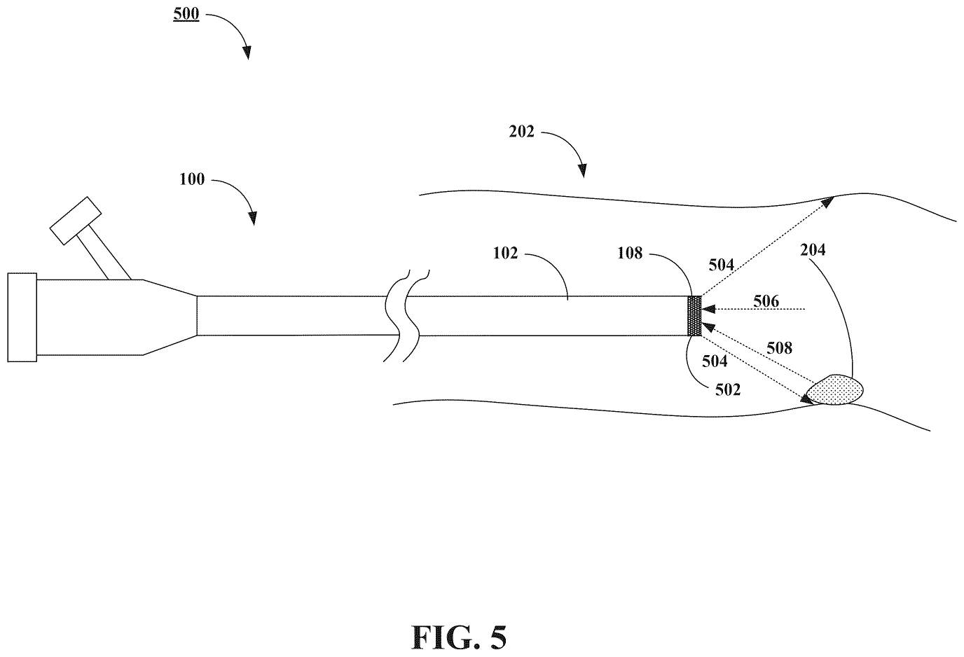

FIG. 5 is a diagram illustrating the example catheter system for blood clot detection 500 that uses ultrasound sensors for the detection of blood clots in accordance with the systems and methods described herein. For example, ultrasound sensors 502 may be built into the catheters, that provide real-time feedback in terms of audio cue when the device is in the vicinity of the clot.

In some example embodiments, an ultrasonic sensor (or sensors) may be a type of electronic equipment that emits ultrasonic sound waves and converts the reflected sound into an electrical signal to determine the distance of a target item. Ultrasonic waves may travel quicker in physiological environments (e.g., passing through liquids and solids) than in air (e.g., typical sounds that humans can hear). The ultrasonic transmitter, which may generate sound using piezoelectric crystals and the ultrasonic receiver, which may encounter the sound after the sound has traveled to and from a target, may be the two main components of an ultrasonic sensor. An ultrasonic sensor may be utilized as a proximity sensor, e.g., proximity to a blood clot.

An ultrasonic sensor may operate by emitting a sound wave at a frequency that is above the range of human hearing. To receive and transmit ultrasonic sound, the sensor's transducer may function as a microphone. Ultrasonic sensors may use a single transducer to send a pulse and receive the echo. The sensor may calculate the distance to a target by measuring the time elapsed between delivering and receiving the ultrasonic pulse.

Ultrasonic sensors 502 may detect the speed of sound within a medium. For example, ultrasound sensors 502 may include a sensor portion and a sound emitting portion. The sound emitting portion of the ultrasound sensors 502 may emit sound 504. Sound 506 may be reflected from blood in the vasculature. Sound 508 may also be reflected from a solid object such as coagulated blood in the vasculature, e.g., a blood clot 204 located in the vasculature. The ultrasound sensors 502 may sense the reflected sound 506, which may have a different velocity depending on what is reflecting the reflected sound 506. For example, blood may typically reflect a speed of sound of approximately 1633 m/s at body temperature through the blood and blood clots may typically reflect a higher speed, e.g., in m/s, when compared to the speed of sound in blood, because sound generally travels faster through a solid than liquid. While the example of FIG. 5 illustrates an ultrasound sensor (or sensors) 502 that includes a sound emitting portion and a sensor, it will be understood that in other embodiments the sound emitting portion and the sensor maybe two distinct devices. Furthermore, it will be understood that some embodiments may include multiple sensors, multiple sound emitting portions or multiple combinations of sensors and sound emitting portions. Additionally, some embodiments may include combinations of individual sensors, individual sound emitting portions, and combined sensors and sound emitting portions.

In an example embodiment, a sound velocity probe may be present at the distal end 110 of the device. The probe may contain an acoustic transducer and reflecting surface. The amount of time for the pulse to move through the medium (e.g., clot or blood) may be used to calculate the speed of sound, either continuously or when prompted. In an example, the speed of sound in a clot may be 1633±4 m/s at 37° C. The speed of sound in blood and the speed of sound in a vessel wall may be predetermined. In this example, the system may signal that clot is present when the ultrasound reads a speed of 1633±4 m/s.

In some embodiments, the precision and sensitivity of ultrasound sensors 502 may be further enhanced by employing advanced signal processing techniques. Such techniques may involve filtering out noise, enhancing the received signals, and utilizing algorithms that specifically identify patterns consistent with blood clot formations. For instance, adaptive filtering may be used to minimize interference from other surrounding tissues, providing a clearer differentiation between the reflected signals from blood and blood clots. Moreover, machine learning models may be trained using a variety of ultrasound readings from different patient demographics to predict the presence of clots with higher accuracy. These technological advancements may assist clinicians in making more informed decisions regarding the location and nature of a clot, potentially reducing the risk of complications and improving patient outcomes.

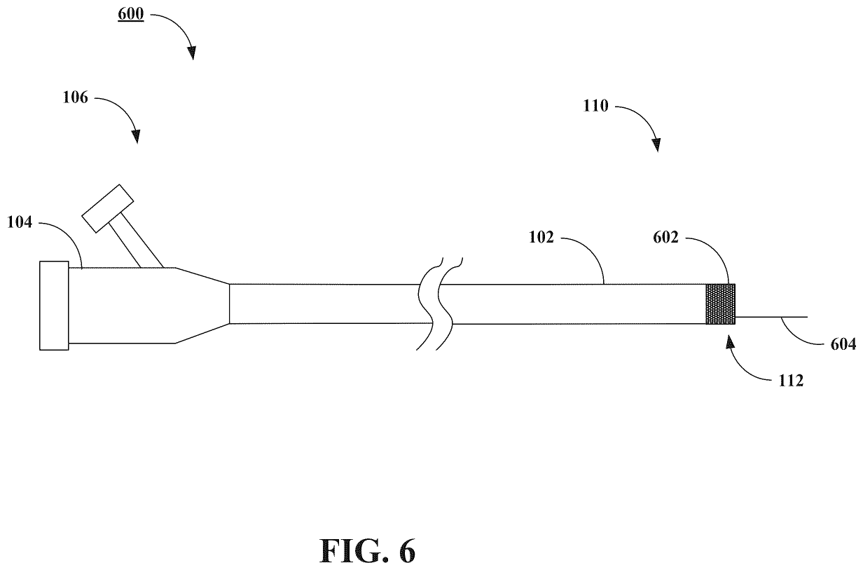

FIG. 6 is a diagram illustrating an example catheter system 600 for blood clot detection including a durometer gauge in accordance with the systems and methods described herein. The example catheter system 600 includes a catheter 102, a y-connector hub 104 situated at a proximal end 106 of the example catheter system 600, and a blood clot sensor (or sensors) 602 at the distal end 110 of the example catheter system 600. For example, the blood clot sensor 602 may be mounted on a tip 112 of the catheter 102. In some embodiments described herein, the blood clot sensor 602 may be a durometer gauge mounted on the tip 112 of the catheter 102. Similarly, in some embodiments described herein, the blood clot sensor 602 may use a mechanism: that includes a retractable atraumatic pin 604 that may be present at the distal end 110 of the device. The user may extend pin 604 to check for a clot. In some embodiments, behind pin 604 may be a spring that compresses a distance proportional to the durometer of the medium. In an example a clot durometer may equal 5-10. Vessel wall durometer may be between 15-20 in some examples. Blood hardness may be 0. These are only examples. In this example, the sensor may signal that a clot is present only when the sensor reads a durometer of values between and including 5-10.

Within the realm of the example catheter system 600, the precise measurement of clot hardness using the durometer gauge, in some embodiments, may offer advantages in clinical settings. For instance, the specificity provided by the durometer gauge may facilitate more accurate differentiation between blood clots, vessel walls, and liquid blood, reducing the likelihood of false positives. The retractable nature of the atraumatic pin 604 may further ensure patient safety, minimizing potential trauma to vessel walls or inadvertent perforations. Moreover, the integration of the durometer gauge with the catheter system 600 may also streamline the procedural workflow in some cases. Real-time feedback on clot presence, as determined by the durometer readings, may guide clinicians in making prompt and informed decisions during interventions. Such prompt decision-making may be crucial, especially in scenarios where rapid clot removal or dissolution is paramount to patient outcomes.

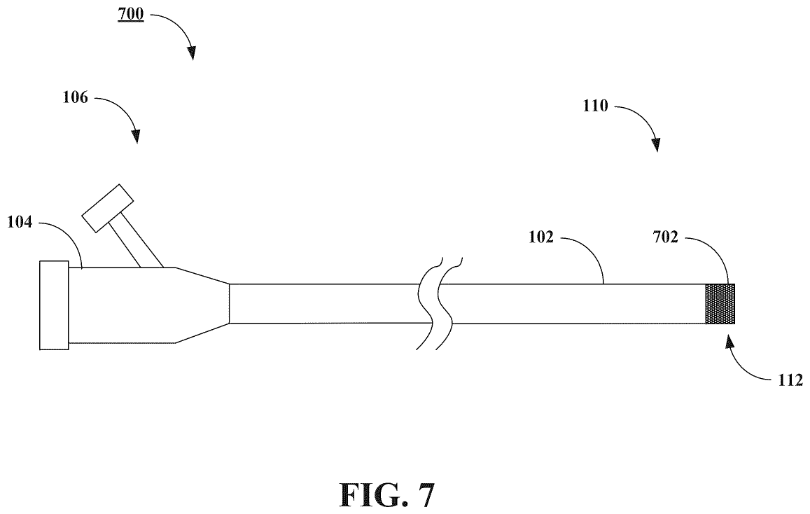

FIG. 7 is a diagram illustrating an example catheter system 700 for blood clot detection including a density meter (e.g., density sensor) in accordance with the systems and methods described herein. Example catheter system 100 includes a catheter 102, a y-connector hub 104 at a proximal end 106 of the example catheter system 100, and a blood clot sensor (or sensors) 702 at the distal end 110 of the example catheter system 100. For example, the blood clot sensor 702 may be mounted on a tip 112 of the catheter 102. In some embodiments described herein, the blood clot sensor 702 may be a density meter mounted on the tip 112 of the catheter 102. Similarly, in some embodiments described herein, the blood clot sensor 702 may use a mechanism that includes a density meter that may be present at the distal end 110 of the device. There are multiple types of density meters on the market that read the density of the medium. Selection of a meter may be based on the following example properties (although other possible properties are also possible). An example clot may have a clot density of 1.08 g/cm³ . The example may have a blood density of 0.994 g/cm³ . The example may include a skin density equal to 1.02 g/cm³. In this example, the system signals that clot is present when it reads a density greater than 1.02 g/cm³.

In the advancement of catheter system technologies, understanding the medium's density within the vasculature may offer insights into the nature of obstructions or potential hazards. The precise differentiation between clots, blood, and surrounding tissues may be used in guiding therapeutic interventions. With respect to FIG. 7, the utility of the density meter as a sensor may greatly enhance the safety and efficiency of clot detection procedures. To ensure accurate measurements, the density meter may employ temperature compensation techniques, as density may be influenced by temperature variations. Furthermore, the density meter sensor may be calibrated regularly to accommodate any systemic drifts or changes over time. This calibration may take into account the physiologic changes in a patient, potential changes in equipment over time, or adjustments made due to innovations in density measurement technology. Such advancements in sensor technologies may not only promote improved patient outcomes but may also reduce procedural time and healthcare costs. By employing density meters in catheter systems, healthcare professionals may gain a more comprehensive understanding of the vascular environment, facilitating informed decisions during procedures.

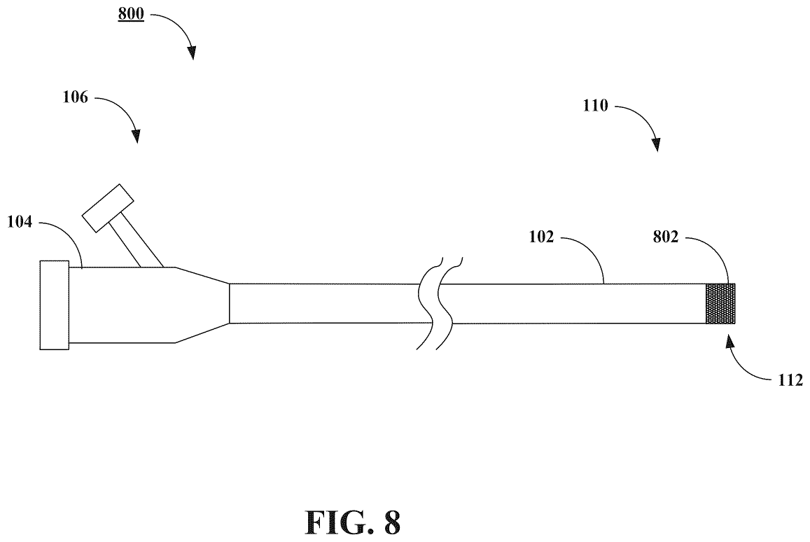

FIG. 8 is a diagram illustrating an example catheter system 800 for blood clot detection including a flow meter in accordance with the systems and methods described herein. The example catheter system 100 includes a catheter 102, a y-connector hub 104 at a proximal end 106 of the example catheter system 100, and a blood clot sensor (or sensors) 802 at the distal end 110 of the example catheter system 100. For example, the blood clot sensor 802 may be mounted on a tip 112 of the catheter 102. In some embodiments described herein, the blood clot sensor 802 may be a flow meter mounted on the tip 112 of the catheter 102. Similarly, in some embodiments described herein, the blood clot sensor 802 may use a mechanism including an inline microfluidic pressure sensor. The flow meter may be present at the distal end 110 of the device. The flow meter may be placed against a medium and increases the flow meter's resistance as blood flow through the measuring unit increases. If the end of the sensor is blocked with clot, there will be no flow and/or a reduced flow through the unit and therefore the signal may be reduced. The system may alert when this drops below a specific value, which may be defined through testing.

The incorporation of a flow meter in the catheter system, as demonstrated in FIG. 8, addresses an aspect of blood clot detection: the impediment or alteration of blood flow due to the presence of a clot. This methodology of detecting blood clots by monitoring variations in flow rates may offer some advantages. For one, it may provide a dynamic approach, wherein changes in real-time blood flow can be instantly recognized. Additionally, this technique may be particularly effective in scenarios where clots are partially occlusive, causing significant reductions in flow but not complete cessation. Regular calibrations of the flow meter may be conducted to help ensure accuracy and reliability of measurements. It should also be noted that while the primary function of the flow meter is to detect clots, it may also provide valuable data on blood flow rates and patterns, which may be indicative of other vascular issues or conditions. As the catheter system evolves, the integration of such multipurpose sensors, like the flow meter, may enhance the safety and precision of vascular interventions.

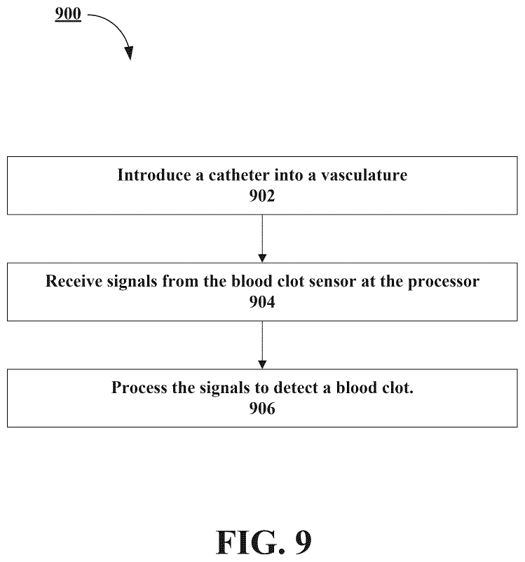

FIG. 9 is a flow diagram illustrating the example method 900 in accordance with the systems and methods described herein. The example method 900 may be a method for blood clot detection. In some embodiments, method 900 may include introducing a catheter into a vasculature (902). The catheter may include a catheter having a proximal end and a distal end, a blood clot sensor (or sensors) coupled to the distal end of the catheter, and a processor configured to process signals from the blood clot sensor. In some embodiments, method 900 may also include receiving signals from the blood clot sensor at the processor (904). In some embodiments, method 900 may also include processing the signals to detect a blood clot (906). In an example embodiment, the blood clot sensor may be a magnetoelastic sensor. In an example embodiment, the blood clot sensor may be a light-based sensor. In an example embodiment, the blood clot sensor may be a light-based sensor.

Method 900, as illustrated in FIG. 9, may be an integral part of the broader scope of blood clot detection systems. In step 902, a catheter is introduced into the vasculature. This catheter might not be a generic catheter, but rather, one designed for this specific method. The catheter may be tailored for precision. For example, the catheter may include a proximal end, a distal end, and a specialized blood clot sensor. In certain configurations, multiple sensors could be coupled to its distal end, ensuring a heightened sensitivity to the potential presence of blood clots. A sub-step may involve selecting the appropriate insertion point for the catheter, taking into account the patient's medical history and the suspected location of the clot. In another sub-step, once the catheter is positioned, a calibration process may be initiated to ensure the sensors are operating at their optimal sensitivity.

As the procedure progresses to step 904, the role of the catheter's integrated technology becomes apparent. The sensors affixed to the catheter may start transmitting signals. These signals, which may contain vital data about blood flow and potential blockages, may be directed towards a dedicated processor. This processor is not just a passive receiver; rather, the processor is designed to interpret the outputs from the blood clot sensor. A sub-step of step 904 may involve the initial filtering of the signals to remove any noise or extraneous data, which ensures the purity of the data being assessed. During another sub-step, the filtered signals may be amplified, if necessary, to further enhance the clarity and precision of the data relayed to the processor.

In step 906, the previously received signals may be processed. The processor delves into detailed processing. The signals may be processed to ascertain the presence or absence of a blood clot. In a sub-step a comparison of the incoming signal data against predefined parameters and thresholds related to blood clot characteristics may be performed. Based on the results of this comparison, the system may generate a diagnostic report indicating the likelihood of a blood clot presence. Method 900 may accommodate a variety of sensors. While some embodiments may employ the magnetoelastic sensor, known for its magnetic responsiveness, other example systems may include one or more light-based sensors. The latter exploits the properties and wavelengths of light to detect anomalies in blood consistency and flow. The emphasis on the light-based sensor, indicated by its repeated mention, underscores its significance in certain versions of the method 900. Some systems may include one or more magnetic sensors and one or more light based sensors, while some example systems may include one or the other.

An example embodiment may include a catheter system for blood clot detection. The system for blood clot detection may include a catheter having a proximal end and a distal end. The system for blood clot detection may also include a blood clot sensor coupled to the distal end of the catheter. Additionally, the system for blood clot detection may include a processor configured to process signals from the blood clot sensor.

The system for blood clot detection may include the blood clot sensor that includes a magnetoelastic sensor. The system for blood clot detection may include a pick-up coil configured to receive magnetic flux from the magnetoelastic sensor and generate a signal based on the magnetic flux. the pick-up coil is internal to the catheter.

In an example embodiment, the pick-up coil is internal to the proximal end of the catheter.

In an example embodiment, the pick-up coil may be external to the catheter.

In an example embodiment, the blood clot sensor may include a light-based sensor. The light-based sensor may include a light sensor configured to measure a wavelength of a reflected beam of light. In an example embodiment, the light-based sensor may include a light source. In an example embodiment, the light source may be part of the light-based sensor.

In an example embodiment, the blood clot sensor may include an ultrasound sensor.

In an example embodiment, the catheter system may output an audio cue based, at least in part, on proximity to a blood clot.

One or more elements or aspects or steps, or any portion(s) thereof, from one or more of any of the systems and methods described herein may be combined with one or more elements or aspects or steps, or any portion(s) thereof, from one or more of any of the other systems and methods described herein and combinations thereof, to form one or more additional implementations and/or claims of the present disclosure.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware.

Reference in the specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment.

Some portions of the detailed description are presented in terms of algorithms and symbolic representations of operations on data bits within a computer memory. These algorithmic descriptions and representations are the methods used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared or otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following disclosure, it is appreciated that throughout the disclosure terms such as “processing,” “computing,” “calculating,” “determining,” “displaying” or the like, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. It will be appreciated that a variety of programming languages may be used to implement the teachings of the disclosure as described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present disclosure has been presented for the purposes of illustration and description. It is not intended to be exhaustive or to limit the present disclosure to the precise form disclosed. Many modifications and variations are possible in light of the above teaching. It is intended that the scope of the present disclosure be limited not by this detailed description, but rather by the claims of this application. As will be understood by those familiar with the art, the present disclosure may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

Furthermore, as will be apparent to one of ordinary skill in the relevant art, the modules, routines, features, attributes, methodologies and other aspects of the present disclosure can be implemented as software, hardware, firmware or any combination of the three. Also, wherever a component, an example of which is a module, of the present disclosure is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming.

Additionally, the present disclosure is in no way limited to implementation in any specific programming language, or for any specific operating system or environment. Accordingly, the disclosure of the present disclosure is intended to be illustrative, but not limiting, of the scope of the present disclosure, which is set forth in the following claims.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.”The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

What is claimed is:

1. A catheter system for blood clot detection, comprising:
a catheter having a proximal end and a distal end;
a blood clot sensor coupled to the distal end of the catheter; and
a processor configured to process signals from the blood clot sensor.

2. The catheter system of claim 1, wherein the blood clot sensor comprises a magnetoelastic sensor.

3. The catheter system of claim 2, further comprising a pick-up coil configured to receive magnetic flux from the magnetoelastic sensor and generate a signal based on the magnetic flux.

4. The catheter system of claim 3, wherein the pick-up coil is internal to the catheter.

5. The catheter system of claim 4, wherein the pick-up coil is internal to the proximal end of the catheter.

6. The catheter system of claim 3, wherein the pick-up coil is external to the catheter.

7. The catheter system of claim 1, wherein the blood clot sensor comprises a light-based sensor.

8. The catheter system of claim 7, wherein the light-based sensor comprises a light sensor configured to measure a wavelength of a reflected beam of light.

9. The catheter system of claim 7, further comprising a light source.

10. The catheter system of claim 9, wherein the light source is part of the light-based sensor.

11. The catheter system of claim 1, wherein the blood clot sensor comprises an ultrasound sensor.

12. The catheter system of claim 1, wherein the catheter system outputs an audio cue based, at least in part, on proximity to a blood clot.

13. The catheter system of claim 1, wherein the blood clot sensor comprises a durometer.

14. The catheter system of claim 1, wherein the blood clot sensor comprises a density sensor.

15. A method for blood clot detection, the method comprising:
introducing a catheter into a vasculature, the catheter including:
a proximal end and a distal end,
a blood clot sensor coupled to the distal end of the catheter, and
a processor configured to process signals from the blood clot sensor,
receiving signals from the blood clot sensor at the processor; and
processing the signals to detect a blood clot.

16. The method of claim 15, wherein the blood clot sensor comprises a magnetoelastic sensor.

17. The method of claim 15, wherein the blood clot sensor comprises a light-based sensor.

18. The method of claim 15, wherein the blood clot sensor comprises an ultrasound sensor.

19. The method of claim 15, wherein the blood clot sensor comprises a durometer.

20. The method of claim 15, wherein the blood clot sensor comprises a density sensor.
